(19) United States
(12) Patent Application Publication
(10) Pub. No.: US 2016/0263160 A1
(43) Pub. Date: Sep. 15, 2016

(54) Mesenchymal Stem Cells Producing Inhibitory RNA for Disease Modification

(71) Applicant: The Regents of the University of California, Oakland, CA (US)

(72) Inventors: Jan A. Nolta, Davis, CA (US); Scott Olson, Davis, CA (US); Louisa Wirthlin, Davis, CA (US)

(21) Appl. No.: 15/162,418

(22) Filed: May 23, 2016

Related U.S. Application Data
(62) Division of application No. 13/260,551, filed on Jan. 18, 2012.
(60) Provisional application No. 61/163,845, filed on Mar. 26, 2009.

(51) Int. Cl. A61K 35/28

(52) U.S. Cl. CPC A61K 35/28

(57) ABSTRACT

Compositions and methods for delivering a siRNA, dsRNA, or miRNA polynucleotide into a target cell comprising contacting the target cell with a mesenchymal stem cell, which mesenchymal stem cell comprises an exogenous DNA sequence expressing the siRNA or dsRNA polynucleotide, thereby delivering the siRNA, dsRNA, or miRNA polynucleotide to the target cell through a cellular protrusion or a microvesicle.

### CROSS-REFERENCE TO RELATED APPLICATIONS

This application is a divisional of U.S. application Ser. No. 13/260,551, filed Jan. 18, 2012, which is a national stage application under 35 U.S.C. §371 of International Application No. PCT/US2010/028712, filed Mar. 25, 2010, which claims the benefit under 35 U.S.C. §119(e) of U.S. Provisional Application No. 61/163,845, filed Mar. 26, 2009, the entire contents of which are hereby incorporated by reference into the present disclosure.

### BACKGROUND

The pathology of Huntington's Disease (HD) is caused by a variable sized polyglutamine (PG) expansion of the protein product of the huntingtin (htt) gene. The best hope for halting HD progression is to reduce or eliminate the mutant htt protein in the affected cells. Direct injection of small interfering RNAs (siRNA) have been shown to be effective at reducing htt levels and ameliorating disease symptoms in animal models (DiFiglia et al. (2007) Proc Natl Acad Sci USA. 104:17204-9 and Wang et al. (2005) Neurosci Res. 53:241-249). Recent data shows that the mutant htt mRNA can be specifically targeted, while sparing the transcript produced by the normal allele (Schwarz et al. (2006) PLoS Genet. 2:e140). The challenge for this technology is to deliver the siRNA into the human brain in a sustained, safe, and effective manner. Direct siRNA delivery is an effective but fleeting answer to a problem. siRNA will not cross the blood-brain barrier for treatment of chronic central nervous system (CNS) diseases like Huntington's, Alzheimer's, Amyotrophic Lateral Sclerosis (ALS) and others. Long term delivery of siRNA to silence the mutant genes, a requirement for treatment of neurodegenerative diseases, remains a critical unsolved issue that is currently thwarting effective therapeutic use. There is a need to develop a method to overcome the siRNA delivery bottleneck, and to develop sustained treatments for neurodegnerative disorders.

### SUMMARY OF THE INVENTION

Applicants have discovered that mesenchymal stem cells, aka marrow stromal cells (MSC) can infuse siRNA and other cellular components directly into damaged cells. Applicants have previously demonstrated, with a decade-long biosafety study, that genetically engineered human MSC are safe. See Bauer et al. Mol Ther. 2008; 16:1308-1315. Phase I/II clinical trials for third party MSC infusions have been conducted now in hundreds of patients without adverse events (early results reviewed in Giordano (2007) J Cell Physiol. 211:27-35 and Salem et al, Stem Cells 2010 in press). Applicants have also shown that MSC can survive integrated into the tissues of immune deficient mice for up to 18 months, while continuing to express the transgene products that they have been genetically engineered to produce (Dao et al (1997) Stem Cells 15:443-453, Bauer et al. (2008) Mol Ther. 16:1308-1315, Meyerrose et al. (2008) Stem Cells 26:1713-22.

Provided is a mesenchymal stem cell comprising, or alternatively consisting essentially of, or yet further consisting of, an exogenous siRNA, miRNA or dsRNA sequence or alternatively or in combination with a DNA sequence encoding a siRNA, miRNA or dsRNA sequence. Also provided is a mesenchymal stem cell comprising, or alternatively consisting essentially of, or yet further consisting of, an exogenous DNA sequence encoding a siRNA, miRNA or dsRNA sequence alone or in combination with the siRNA, miRNA or dsRNA sequence. In a further aspect, each of the MSC described above can establish a cellular protrusion with a target cell thereby delivering the polynucleotide and/or the siRNA, miRNA or dsRNA to the target cell. In a further aspect, the MSC can deliver the polynucleotide and/or the siRNA, miRNA or dsRNA or the polynucleotide encoding it via a microvesicle to the target cell. In a further aspect, the polynucleotide and/or siRNA, miRNA or dsRNA is delivered to the target cell by any method which excludes a gap junction via connexin. In one aspect, the mesenchymal stem cell is an isolated mesenchymal stem cell and in another aspect the cell is present in tissue isolated from a suitable subject, such as lipoaspirate or bone marrow sample.

Also provided is a method for delivering a siRNA, miRNA or dsRNA polynucleotide into a target cell comprising or alternatively consisting essentially of, or yet further consisting of, contacting the target cell with a mesenchymal stem cell, which mesenchymal stem cell comprises an exogenous DNA sequence expressing the siRNA, miRNA or dsRNA polynucleotide, thereby delivering the siRNA, miRNA or dsRNA polynucleotide to the target cell. Without being bound by theory, the delivery can independently or in combination occur by or through a cellular protrusion and/or via a microvesicle. In a further aspect, the polynucleotide and/or siRNA or dsRNA is delivered to the target cell by any method which excludes a gap junction via connexin. In one aspect, the mesenchymal stem cell is an isolated mesenchymal stem cell and in another aspect the cell is present in tissue isolated from a suitable subject, such as lipoaspirate or bone marrow sample.

Also provided is a method for treating a genetic condition mediated by the presence of a mutated allele in a subject, for example Huntington's disease in a patient by administering to the patient the MSC as described above or a composition comprising, or alternatively consisting essentially of, or yet further consisting of, a mesenchymal stem cell, wherein the polynucleotide and/or the siRNA, miRNA or dsRNA is directed at a mutant Htt gene, and can deliver the siRNA, miRNA or dsRNA to a target nerve cell in the patient. Without being bound by theory, in one aspect, the MSC of the invention is one in which the polynucleotide and/or siRNA, miRNA or dsRNA is independently or collectively delivered through a cellular protrusion and/or a microvesicle, thereby treating the disease. In a further aspect, the polynucleotide and/or siRNA, miRNA or dsRNA is delivered to the target cell by any method which excludes a gap junction via connexin. In one aspect, the mesenchymal stem cell is an isolated mesenchymal stem cell and in another aspect the cell is present in tissue isolated from a suitable subject, such as lipoaspirate or bone marrow sample.

Therefore, this invention provides compositions and methods to deliver a siRNA, miRNA or dsRNA to a target organ such as the brain in a sustained, safe, and effective manner using the methods and compositions as described herein.

### BRIEF DESCRIPTION OF THE FIGURES

FIG. 1 shows representative field from co-cultures of Alexafluor 547 labeled siRNA transfected MSC and GFP⁺ MSC after 96 hours of incubation. Shown: eGFP-labeled MSC that has had alexa-fluor-labeled anti mutant htt siRNA (as indicated by circles around the bright spots) transferred into it from an adjacent, non-GFP MSC. Color merged z-projection.

FIG. 2 shows co-cultures of Alexafluor 547 labeled siRNA transfected MSC (as indicated by circles around bright spots or area) and GFP⁺ MSC after 24 hours of incubation. Panel A shows an aximum intensity z-projection of GFP channel alone. Panel B shows the maximum intensity z-projection of Alexafluor 547 labeled siRNA channel alone Panel D is a color merged maximum intensity z-projection. Panel F. is a zoom of Panel D to more easily see the presence of transferred siRNA throughout target cell.

FIG. 3A shows IV injected Human MSC seeded to different tissues in irradiated mice. The human cells are visualized by the stains indicated by circles around them for endogenous levels of the GUSB enzyme, which is absent in NOD/SCID/MPSVII mice. FIG. 3B (Panels A through C) shows MSC-produced Beta-glucuronidase (GUSB) distribution following transplantation. In Panel A, there is no demonstrable GUSB activity in the liver of a 4-month-old NOD/SCID/MPSVII mouse that did not undergo transplantation. In Panel B, low numbers of GUSB-positive cells (red stain) are observed in the liver of a 4-month-old MPSVII mouse that received a transplant of control MSC expressing enhanced green fluorescent protein (MSC-eGFP). The number of GUSB-positive cells is in the same range as the number of human cells detected by quantitative polymerase chain reaction. In Panel C, nearly every cell in the liver of a 4-month-old MPSVII mouse that received a transplant of MSCs engineered to secrete GUSB is positive for the vector product.

### DETAILED DESCRIPTION

Throughout this disclosure, various publications, patents and published patent specifications are referenced by an identifying citation. The disclosures of these publications, patents and published patent specifications are hereby incorporated by reference in their entirety into the present disclosure.

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; Animal Cell Culture (R. I. Freshney, ed. (1987)); Zigova, Sanberg and Sanchez-Ramos, eds. (2002) Neural Stem Cells.

All numerical designations, e.g., pH, temperature, time, concentration, and molecular weight, including ranges, are approximations which are varied (+) or (−) by increments of 0.1 or 1 where appropriate. It is to be understood, although not always explicitly stated that all numerical designations are preceded by the term “about”. The term “about” also includes the exact value “X” in addition to minor increments of “X” such as “X+0.1 or 1” or “X−0.1 or 1,” where appropriate. It also is to be understood, although not always explicitly stated, that the reagents described herein are merely exemplary and that equivalents of such are known in the art.

### Definitions

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

The term “isolated” as used with respect to cells, in particular stem cells, such as mesenchymal stem cells, refers to cells separated from other cells or tissue that are present in the natural tissue in the body.

A “subject,” “individual” or “patient” is used interchangeably herein and refers to a vertebrate, for example a primate, a mammal or preferably a human. Mammals include, but are not limited to equines, canines, bovines, ovines, murines, rats, simians, humans, farm animals, sport animals and pets.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Amplify” “amplifying” or “amplification” of a polynucleotide sequence includes methods such as traditional cloning methodologies, PCR, ligation amplification (or ligase chain reaction, LCR) or other amplification methods. These methods are known and practiced in the art. See, e.g., U.S. Patent Nos. 4,683,195 and 4,683,202 and Innis et al. (1990) Mol. Cell Biol. 10(11):5977-5982 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “genotype” refers to the specific allelic composition of an entire cell, a certain gene or a specific polynucleotide region of a genome, whereas the term “phenotype” refers to the detectable outward manifestations of a specific genotype.

As used herein, the term “gene” or “recombinant gene” refers to a nucleic acid molecule comprising an open reading frame and including at least one exon and (optionally) an intron sequence. A gene may also refer to a polymorphic or a mutant form or allele of a gene.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on May 21, 2008. Biologically equivalent polynucleotides are those having the specified percent homology and encoding a polypeptide having the same or similar biological activity.

The term “an equivalent nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, or nucleic acid-nucleic acid in nature.

“Hybridization” refers to a reaction in which one or more polynucleotides react to form a hybridization complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A non-limiting example of a physiological condition is the temperature, ionic strength, pH and concentration of Mg²⁺ normally found in a cell.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

The term “mismatches” refers to hybridized nucleic acid duplexes which are not 100% homologous. The lack of total homology may be due to deletions, insertions, inversions, substitutions or frameshift mutations.

As used herein, the term “oligonucleotide” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

As used herein, the term “carrier” encompasses any of the standard carriers, such as a phosphate buffered saline solution, buffers, water, and emulsions, such as an oil/water or water/oil emulsion, and various types of wetting agents. The compositions also can include stabilizers and preservatives. For examples of carriers, stabilizers and adjuvants, see Sambrook and Russell (2001), supra. Those skilled in the art will know many other suitable carriers for binding polynucleotides, or will be able to ascertain the same by use of routine experimentation. In one aspect of the invention, the carrier is a buffered solution such as, but not limited to, a PCR buffer solution.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection, sometimes called transduction), transfection, transformation or various other protein-based or lipid-based gene delivery complexes) as well as techniques facilitating the delivery of “naked” polynucleotides (such as electroporation, “gene gun” delivery and various other techniques used for the introduction of polynucleotides). Unless otherwise specified, the term transfected, transduced or transformed may be used interchangeably herein to indicate the presence of exogenous polynucleotides or the expressed polypeptide therefrom in a cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

The term “express” refers to the production of a gene product. In some embodiments, the gene product is a polypeptide or protein. In some embodiments, the gene product is a mRNA, a tRNA, a rRNA, a miRNA, a dsRNA, or a siRNA.

A cell that “stably expresses” an exogenous polypeptide is one that continues to express a polypeptide encoded by an exogenous gene introduced into the cell either after replication if the cell is dividing or for longer than a day, up to about a week, up to about two weeks, up to three weeks, up to four weeks, for several weeks, up to a month, up to two months, up to three months, for several months, up to a year or more.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, retroviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski, et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels are described and exemplified herein.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in M. MacPherson et al. (1991) PCR: A Practical Approach, IRL Press at Oxford University Press. All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra. The primers may optionall contain detectable labels and are exemplified and described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluoresecence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

The phrase “solid support” refers to non-aqueous surfaces such as “culture plates” “gene chips” or “microarrays.” Such gene chips or microarrays can be used for diagnostic and therapeutic purposes by a number of techniques known to one of skill in the art. In one technique, oligonucleotides are attached and arrayed on a gene chip for determining the DNA sequence by the hybridization approach, such as that outlined in U.S. Pat. Nos. 6,025,136 and 6,018,041. The polynucleotides of this invention can be modified to probes, which in turn can be used for detection of a genetic sequence. Such techniques have been described, for example, in U.S. Patent Nos. 5,968,740 and 5,858,659. A probe also can be attached or affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarrays” and similar technologies are known in the art. Examples of such include, but are not limited to, LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarry system that can dispense from 12 to 64 spots onto multiple glass slides (Intelligent Bio-Instruments); Molecular Biology Workstation and NanoChip (Nanogen, Inc.); a microfluidic glass chip (Orchid Biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or a “microarrays” are also described in U.S. Patent Publication Nos.: 2007/0111322; 2007/0099198; 2007/0084997; 2007/0059769 and 2007/0059765 and U.S. Pat. Nos. 7,138,506; 7,070,740 and 6,989,267.

In one aspect, “gene chips” or “microarrays” containing probes or primers homologous to a polynucleotide described herein are prepared. A suitable sample is obtained from the patient, extraction of genomic DNA, RNA, protein or any combination thereof is conducted and amplified if necessary. The sample is contacted to the gene chip or microarray panel under conditions suitable for hybridization of the gene(s) or gene product(s) of interest to the probe(s) or primer(s) contained on the gene chip or microarray. The probes or primers may be detectably labeled thereby identifying the sequence(s) of interest. Alternatively, a chemical or biological reaction may be used to identify the probes or primers which hybridized with the DNA or RNA of the gene(s) of interest. The genotypes or phenotype of the patient is then determined with the aid of the aforementioned apparatus and methods.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

For topical use, the pharmaceutically acceptable carrier is suitable for manufacture of creams, ointments, jellies, gels, solutions, suspensions, etc. Such carriers are conventional in the art, e.g., for topical administration with polyethylene glycol (PEG). These formulations may optionally comprise additional pharmaceutically acceptable ingredients such as diluents, stabilizers, and/or adjuvants.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker, e.g. myosin or actin or the expression of a gene or protein, e.g. a calcium handling protein, a t-tubule protein or alternatively, a calcium pump protein. In another aspects, the substantially homogenous population have a decreased (e.g., less than about 95%, or alternatively less than about 90%, or alternatively less than about 80%, or alternatively less than about 75%, or alternatively less than about 70%, or alternatively less than about 65%, or alternatively less than about 60%, or alternatively less than about 55%, or alternatively less than about 50%) of the normal level of expression than the wild-type counterpart cell or tissue.

A “neurodegenerative disease” is a condition in which cells of the brain and spinal cord are lost. Examples of neurodegenerative diseases include, but are not limited to, Huntington's disease, ALS and multiple sclerosis. The brain and spinal cord are composed of neurons that do different functions such as controlling movements, processing sensory information, and making decisions. Cells of the brain and spinal cord are not readily regenerated en masse, so excessive damage can be devastating. Neurodegenerative diseases result from deterioration of neurons or their myelin sheath which over time will lead to dysfunction and disabilities resulting from this.

A “subject” of diagnosis or treatment is a cell or a mammal, including a human. Non-human animals subject to diagnosis or treatment include, for example, simians, murines, guinea pigs, canines, such as dogs, leporids, such as rabbits, livestock, such as bovine or porcine, sport animals, and pets.

An “effective amount” is an amount sufficient to effect beneficial or desired results. An effective amount can be administered in one or more administrations, applications or dosages and can be empirically determined by those of skill in the art.

A “control” is an alternative subject or sample used in an experiment for comparison purpose. A control can be “positive” or “negative”. For example, where the purpose of the experiment is to determine a correlation of a mutated allele with a particular phenotype, it is generally preferable to use a positive control (a sample from a subject, carrying such mutation and exhibiting the desired phenotype), and a negative control (a subject or a sample from a subject lacking the mutated allele and lacking the phenotype).

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

A neoplasm is an abnormal mass or colony of cells produced by a relatively autonomous new growth of tissue. Most neoplasms arise from the clonal expansion of a single cell that has undergone neoplastic transformation. The transformation of a normal to a neoplastic cell can be caused by a chemical, physical, or biological agent (or event) that directly and irreversibly alters the cell genome. Neoplastic cells are characterized by the loss of some specialized functions and the acquisition of new biological properties, foremost, the property of relatively autonomous (uncontrolled) growth. Neoplastic cells pass on their heritable biological characteristics to progeny cells.

The past, present, and future predicted biological behavior, or clinical course, of a neoplasm is further classified as benign or malignant, a distinction of great importance in diagnosis, treatment, and prognosis. A malignant neoplasm manifests a greater degree of autonomy, is capable of invasion and metastatic spread, may be resistant to treatment, and may cause death. A benign neoplasm has a lesser degree of autonomy, is usually not invasive, does not metastasize, and generally produces no great harm if treated adequately.

Cancer is a generic term for malignant neoplasms. Anaplasia is a characteristic property of cancer cells and denotes a lack of normal structural and functional characteristics (undifferentiation).

A tumor is literally a swelling of any type, such as an inflammatory or other swelling, but modem usage generally denotes a neoplasm. The suffix “-oma” means tumor and usually denotes a benign neoplasm, as in fibroma, lipoma, and so forth, but sometimes implies a malignant neoplasm, as with so-called melanoma, hepatoma, and seminoma, or even a non-neoplastic lesion, such as a hematoma, granuloma, or hamartoma. The suffix “-blastoma” denotes a neoplasm of embryonic cells, such as neuroblastoma of the adrenal or retinoblastoma of the eye.

Histogenesis is the origin of a tissue and is a method of classifying neoplasms on the basis of the tissue cell of origin. Adenomas are benign neoplasms of glandular epithelium. Carcinomas are malignant tumors of epithelium. Sarcomas are malignant tumors of mesenchymal tissues. One system to classify neoplasia utilizes biological (clinical) behavior, whether benign or malignant, and the histogenesis, the tissue or cell of origin of the neoplasm as determined by histologic and cytologic examination. Neoplasms may originate in almost any tissue containing cells capable of mitotic division. The histogenetic classification of neoplasms is based upon the tissue (or cell) of origin as determined by histologic and cytologic examination.

“Suppressing” tumor growth indicates a growth state that is curtailed compared to growth without any therapy. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and “suppressing” tumor growth indicates a growth state that is curtailed when stopping tumor growth, as well as tumor shrinkage.

siRNA, dsRNA, miRNA

“RNA interference” (RNAi) refers to sequence-specific or gene specific suppression of gene expression (protein synthesis) that is mediated by short interfering RNA (siRNA).

“Short interfering RNA” (siRNA) refers to double-stranded RNA molecules (dsRNA), generally, from about 10 to about 30 nucleotides in length that are capable of mediating RNA interference (RNAi), or 11 nucleotides in length, 12 nucleotides in length, 13 nucleotides in length, 14 nucleotidesw in length, 15 nucleotidesw in length, 16 nucleotidesw in length, 17 nucleotides in length, 18 nucleotidesw in length, 19 nucleotidesw in length, 20 nucleotides in length, 21 nucleotidesw in length, 22 nucleotidesw in length, 23 nucleotidesw in length, 24 nucleotides in length, 25 nucleotidesw in length, 26 nucleotidesw in length, 27 nucleotidesw in length, 28 nucleotides in length, or 29 nucleotides in length. As used herein, the term siRNA includes short hairpin RNAs (shRNAs). A siRNA directed to a gene or the mRNA of a gene may be a siRNA that recognizes the mRNA of the gene and directs a RNA-induced silencing complex (RISC) to the mRNA, leading to degradation of the mRNA. A siRNA directed to a gene or the mRNA of a gene may also be a siRNA that recognizes the mRNA and inhibits translation of the mRNA.

“Double stranded RNA” (dsRNA) refer to double stranded RNA molecules that may be of any length and may be cleaved intracellularly into smaller RNA molecules, such as siRNA. In cells that have a competent interferon response, longer dsRNA, such as those longer than about 30 base pair in length, may trigger the interferon response. In other cells that do not have a competent interferon response, dsRNA may be used to trigger specific RNAi.

A siRNA can be designed following procedures known in the art. See, e.g., Dykxhoorn, D. M. and Lieberman, J. (2006) “Running Interference: Prospects and Obstacles to Using Small Interfering RNAs as Small Molecule Drugs,” Annu. Rev. Biomed. Eng. 8:377-402; Dykxhoorn, D. M. et al. (2006) “The silent treatment: siRNAs as small molecule drugs,” Gene Therapy, 13:541-52; Aagaard, L. and Rossi, J. J. (2007) “RNAi therapeutics: Principles, prospects and challenges,” Adv. Drug Delivery Rev. 59:75-86; de Fougerolles, A. et al. (2007) “Interfering with disease: a progress report on siRNA-based therapeutics,” Nature Reviews Drug Discovery 6:443-53; Krueger, U. et al. (2007) “Insights into effective RNAi gained from large-scale siRNA validation screening,” Oligonucleotides 17:237-250; U.S. Patent Application Publication No.: 2008/0188430; and U.S. Patent Application Publication No.: 2008/0249055.

Delivery of siRNA to a mesenchymal stem cell to generate the cell of this invention can be made with methods known in the art. See, e.g., Dykxhoorn, D. M. and Lieberman, J. (2006) “Running Interference: Prospects and Obstacles to Using Small Interfering RNAs as Small Molecule Drugs,” Annu. Rev. Biomed. Eng. 8:377-402; Dykxhoorn, D. M. et al. (2006) “The silent treatment: siRNAs as small molecule drugs,” Gene Therapy, 13:541-52; Aagaard, L. and Rossi, J. J. (2007) “RNAi therapeutics: Principles, prospects and challenges,” Adv. Drug Delivery Rev. 59:75-86; de Fougerolles, A. et al. (2007) “Interfering with disease: a progress report on siRNA-based therapeutics,” Nature Reviews Drug Discovery 6:443-53; Krueger, U. et al. (2007) “Insights into effective RNAi gained from large-scale siRNA validation screening,” Oligonucleotides 17:237-250; U.S. Patent Application Publication No.: 2008/0188430; and U.S. Patent Application Publication No.: 2008/0249055.

A siRNA may be chemically modified to increase its stability and safety. See, e.g. Dykxhoorn, D. M. and Lieberman, J. (2006) “Running Interference: Prospects and Obstacles to Using Small Interfering RNAs as Small Molecule Drugs,” Annu. Rev. Biomed. Eng. 8:377-402 and U.S. Patent Application Publication No.: 2008/0249055.

microRNA or miRNA are single-stranded RNA molecules of 21-23 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

A siRNA vector, dsRNA vector or miRNA vector as used herein, refers to a plasmid or viral vector comprising a promoter regulating expression of the RNA. “siRNA promoters” or promoters that regulate expression of siRNA, dsRNA, or miRNA are known in the art, e.g., a U6 promoter as described in Miyagishi and Taira (2002) Nature Biotech. 20:497-500, and a H1 promoter as described in Brummelkamp et al. (2002) Science 296:550-3.

### Stem Cells

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Non-limiting examples of embryonic stem cells are the HES2 (also known as ES02) cell line available from ESI, Singapore and the H1 (also know as WA01) cell line available from WiCells, Madison, Wis. Pluripotent embryonic stem cells can be distinguished from other types of cells by the use of marker including, but not limited to, Oct-4, alkaline phosphatase, CD30, TDGF-1, GCTM-2, Genesis, Germ cell nuclear factor, SSEA1, SSEA3, and SSEA4.

A “mesenchymal stem cell” or MSC, is a multipotent stem cell that can differentiate into a variety of cell types. The designation MSC also refers to the term “marrow stromal cell”. Cell types that MSCs have been shown to differentiate into in vitro or in vivo include osteoblasts, chondrocytes, myocytes, and adipocytes. Mesenchyme is embryonic connective tissue that is derived from the mesoderm and that differentiates into hematopoietic and connective tissue, whereas MSCs do not differentiate into hematopoietic cells. Stromal cells are connective tissue cells that form the supportive structure in which the functional cells of the tissue reside. While this is an accurate description for one function of MSCs, the term fails to convey the relatively recently-discovered roles of MSCs in repair of tissue. Applicants have described methods to isolate, propagate, and genetically engineer marrow stromal cells/mesenchymal stem cells (MSC) for over two decades (reviewed in Nolta, Genetic Engineering of Mesenchymal Stem Cells, Springer 2006). Methods to isolate such cells, propagate and differentiate such cells are known in the technical and patent literature, e.g., U.S. Patent Application Publication Nos: 2007/0224171, 2007/0054399, 2009/0010895, which are incorporated by reference in their entirety.

A “neural or neuronal stem cell” as used herein refers to a cell that has the ability to self-replicate and give rise to multiple specialized cell types of the nervous system. In some aspect, a neural stem cell is a multipotential neural stem cell in the subventricular zone (SVZ) of the forebrain lateral ventricle (LV).

A clone or “clonal population” is a line of cells that is genetically identical to the originating cell; in this case, a stem cell. A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a farther stage of cell differentiation. Progenitor cells are often found in adult organisms, they act as a repair system for the body. Examples of progenitor cells include, but are not limited to, satellite cells found in muscles, intermediate progenitor cells formed in the subventricular zone, bone marrow stromal cells, periosteum progenitor cells, pancreatic progenitor cells and angioblasts or endothelial progenitor cells. Examples of progenitor cells may also include, but are not limited to, an ependymal cell and a neural stem cell from the forebrain lateral ventricle (LV).

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Clonal proliferation” refers to the growth of a population of cells by the continuous division of single cells into two identical daughter cells and/or population of identical cells.

As used herein, the “lineage” of a cell defines the heredity of the cell, i.e. its predecessors and progeny. The lineage of a cell places the cell within a hereditary scheme of development and differentiation.

A derivative of a cell or population of cells is a daughter cell of the isolated cell or population of cells. Derivatives include the expanded clonal cells or differentiated cells cultured and propagated from the isolated stem cell or population of stem cells. Derivatives also include already derived stem cells or population of stem cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

As used herein, a “pluripotent cell” defines a less differentiated cell that can give rise to at least two distinct (genotypically and/or phenotypically) further differentiated progeny cells. In another aspect, a “pluripotent cell” includes a Induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e. Oct-3/4; the family of Sox genes, i.e. Sox1, Sox2, Sox3, Sox15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi K. et al. (2007) Cell advance online publication 20 Nov. 2007; Takahashi K. & Yamanaka S. (2006) Cell 126: 663-76; Okita K. et al. (2007) Nature 448:260-262; Yu, J. et al. (2007) Science advance online publication 20 Nov. 2007; and Nakagawa, M. et al. (2007) Nat. Biotechnol. Advance online publication 30 Nov. 2007.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller (2006) The Promise of Stem Cells for Neural Repair, Brain Res. Vol. 1091 (1):258-264; Pluchino et al. (2005) Neural Stem Cells and Their Use as Therapeutic Tool in Neurological Disorders, Brain Res. Brain Res. Rev., Vol. 48(2):211-219; and Goh, et al. (2003) Adult Neural Stem Cells and Repair of the Adult Central Nervous System, J. Hematother. Stem Cell Res., Vol. 12(6):671-679.

A population of cells intends a collection of more than one cell that is identical (clonal) or non-identical in phenotype and/or genotype.

“Cellular protrusion” as used herein, refers to a cell-to-cell contact that does not involve a connexin protein or a gap junction type connection. In one aspect, a cellular protrusion is a cytoplasmic extension or broad areas of cellular contact as observed between a MSC and a skin fibroblast cell as described by Applicants in Spees et al. (2006) PNAS 103(5):1283-8. In another aspect, a cellular protrusion is a tunneling nanotube formed between a MSC and a cardiomyocyte in co-culture observed in Plotnikov et al. (2008) J. Cell. Mol. Med. 12(5A):1622-31. In some embodiments, a cellular protrusion is a thin, elongated, active filopodia and lamellipodia, a cytoneme, a cytoneme-like protrusion, an apical peripodial extension, a myopodia, a myopdia-like protrusion, a cellular extension, or an apical and lateral cell protrusion as reviewed in Gurke et al. (2008) Histochem. Cell Biol. 129:539-50.

“Microvesicles” are fragments of plasma membrane ranging from 100 nm to 700 nm shed from almost all cell types during activation or apoptosis. They originate directly from the plasma membrane of the cell and reflect the antigenic content of the cells which they originate from.

### MODES FOR CARRYING OUT THE DISCLOSURE

The pathology of Huntington's Disease (HD) is caused by a variable sized polyglutamine (PG) expansion of the protein product of the huntingtin (htt) gene. The Htt gene is located on the short arm of chromosome 4. Htt contains a sequence of three DNA bases—cytosine-adenine-guanine (CAG)—repeated multiple times, known as a trinucleotide repeat. Generally, people have less than 27 repeated glutamines. Htt with fewer than 36 glutamines results in production of the cytoplasmic protein called huntingtin. However, a sequence of 36 or more glutamines results in the production of a form of Htt which has different characteristics. This altered form, called mutant Htt or more commonly mHtt, increases the rate of neuronal decay in certain types of neurons and the brain regions which have a higher proportion or dependency on them. Generally, the number of CAG repeats is related to how much this process is affected, and correlates with age at onset and the rate of progression of symptoms. For example, 36-39 repeats result in much later onset and slower progression of symptoms than the mean, such that some individuals may die of other causes before they even manifest symptoms of Huntington disease; this is termed “reduced penetrance”. With very large repeat counts, HD can occur under the age of 20 years, when it is then referred to as juvenile HD, akinetic-rigid, or Westphal variant HD; this accounts for about 7% of HD carriers.

The best hope for halting HD progression is to reduce or eliminate the mutant htt protein in the affected cells. Small interfering RNAs (siRNA) have been shown to be effective at reducing htt levels and ameliorating disease symptoms in animal models (DiFiglia et al. (2007) Proc Natl Acad Sci USA. 104:17204-17209; Wang et al. (2005) Neurosci Res. 53:241-249). New data shows that the mutant htt mRNA can be specifically targeted, while sparing the transcript produced by the normal allele (Schwarz (2006) PLoS Genet. 2:e140). The challenge for this technology is to deliver the siRNA into the human brain in a sustained, safe, and effective manner. Direct siRNA delivery is an effective but fleeting answer to a problem. siRNA will not cross the blood-brain barrier for treatment of chronic central nervous system (CNS) diseases like Huntington's, Alzheimer's, ALS and others. Long term delivery of siRNA to silence the mutant genes, a requirement for treatment of neurodegenerative diseases, remains a critical unsolved issue that is currently thwarting effective therapeutic use. The current invention addresses the siRNA delivery bottleneck, and develops sustained treatments for neurodegnerative disorders and other diseases medicated by genes or genetic variations or mutations of genes.

This invention uses human mesenchymal stem cells (MSC) engineered to continually deliver anti-mutant htt siRNA into damaged or at-risk neurons in the brain. Applicants have used MSC, “the paramedics of the body,” over the past 21 years to safely and effectively deliver many molecules systemically and to multiple organs, including neural tissue, in vivo (Dao et al. (1997) Stem Cells. 15:443-454; Meyerrose et al. (2007) Stem Cells. 25:220-227; Meyerrose et al. (2008) Stem Cells. 26:1713-1722; Nolta et al. (1994) Blood. 83:3041-3051; Tsark et al. (2001) J Immunol. 166:170-181; Wang et al. (2003) Blood. 101 (10) 4201-4208). It has been reported in these publications that MSC/marrow stromal cells robustly produce products for delivery into other cells in vivo, in a sustained manner. Applicants have also shown that MSC infused siRNA and other cellular components directly into damaged cells. Using this delivery method, frequent siRNA readministration would not be necessary. Using this approach, an adult stem cell therapy-based delivery strategy is developed that could have far-reaching impact into any neurodegenerative disorder where a toxic mutant protein must be decreased.

It is contemplated that a clinical trial will be conducted to use intra-striatal injection of anti-mutant htt siRNA engineered MSC to treat early-stage HD, to prevent further neuronal loss and debilitation. A decade-long biosafety study has just been finished by the Applicants to show that genetically engineered human MSC's are safe (Bauer et al. (2008) Mol Ther. 16:1308-1315). Phase I/II clinical trials for third party MSC infusions have been conducted now in hundreds of patients without adverse events (early results reviewed in Giordano et al. (2007) J Cell Physiol. 211:27-35). MSC's have been successfully infused into the brains of patients with ALS, without adverse events (Mazzini et al. (2003) Amyotroph Lateral Scler Other Motor Neuron Disord. 4:158-161). Since HD patients unfortunately have few other options, the benefit to risk ratio for this future trial is extremely high.

Different populations of stem cells have been described to contribute to the regeneration of muscle, liver, heart, and vasculature, although the mechanisms by which this is accomplished are still not well understood. Stem cells are known, however, to secrete a variety of cytokines and growth factors that have both paracrine and autocrine activities. A theory of tissue repair and regeneration by adult MSC is that the mechanism of action is based upon the innate functions of the stem cells: the injected stem cells home to the injured area, in particular to hypoxic, apoptotic, or inflamed areas, and release trophic factors that hasten endogenous repair. These secreted bioactive factors suppress the local immune system, enhance angiogenesis, inhibit fibrosis and apoptosis, and stimulate recruitment, retention, mitosis, and differentiation of tissue-residing stem cells. These trophic effects are distinct from the direct differentiation of stem cells into the tissue to be regenerated. MSC have been shown to contribute to the recovery of tissues in multiple injury models such as myocardial infarction (Laflamme & Murry (2005) Nat Biotechnol. 23:845-856), stroke model (Chen et al. (2003) J Neurosci Res. 73:778-786; Li et al. (2005) Glia. 49:407-417), meniscus injury model (Murphy et al. (2003) Arthritis Rheum. 48:3464-3474), and hind limb ischemia (Rosova et al. (2008) Stem Cells 26:2173-2182). Trophic factor secretion and overall augmentation of tissue regeneration have been shown in a cardiac infarction model (Gnecchi et al. (2006) Faseb J. 20:661-669), and the secretion of multiple angiogenic-stimulating cytokines including HGF, FGF-2, insulin growth factor-I (IGF-I), and vascular endothelial growth factor (VEGF) have been detected in MSC-conditioned medium. It is discovered that a complex set of trophic factors secreted by the MSC appears to significantly contribute toward repair of damaged tissues in vivo, through stimulating angiogenesis and decreasing apoptosis.

The trophic effects of MSC in the brain include promoting endogenous neuronal growth through secreted growth factors, secreting anti-apoptotic factors, and regulating inflammation. In mice that have a deficiency of acid sphingomyelinase, the transplantation of MSC delayed the onset of development of neurological abnormalities and significantly extended their lifespan (Chen et al. (2001) Stroke. 32:1005-1011; Jin et al. (2002) J Clin Invest. 109:1183-1191). Due to the promise of MSC-secreted survival factors reducing cell death, Mazzini et al. initiated a clinical study to verify the efficacy of MSC transplantation in amyotrophic lateral sclerosis (ALS) patients (Mazzini et al. (2003) Amyotroph Lateral Scler Other Motor Neuron Disord. 4:158-161). ALS causes a loss of motor neurons leading to a progressive and fatal decline in muscle functionality. Seven patients with ALS, who already had severe functional impairment of their legs, were enrolled in the MSC clinical trial Expanded MSC were transplanted into the patients' spinal cords. No adverse events were caused by the treatments. Three months after cell implantation, a trend toward a slowing down of the decline in muscular strength was observed in the legs of four patients (Mazzini et al. (2003) Amyotroph Lateral Scler Other Motor Neuron Disord. 4:158-161). Since a randomized study was not done, the results are in no way definitive, but they do show that MSC infusion into the cerebrospinal fluid could be tolerated without adverse events in patients with one form of a neurodegenerative disorder. This invention not only utilizes the innate trophic effects of the MSC, but also uses them as delivery vehicles to infuse siRNA designed to attack the mutant RNA species responsible for the neurodegenerative disorder in HD.

There are a number of good transgenic mouse models to overexpress different forms of the mutant htt protein, with variable length repeats (see, e.g., Heng et al. (2008) Neurobiol Dis. 32:1-9; Ramaswamy et al. (2007) Ilar J. 48:356-373). However, human cells cannot be reliably transplanted into these strains, which have full immune competence. Therefore a model that is created by lentiviral transduction of murine neurons using lentiviral vectors is used, as initially described by de Almeida et al. (2002) J Neurosci. 22:3473-3483. The authors performed stereotactic injection into the left and right striatum to examine the effects of lentiviral delivery of a truncated form of the human htt protein that had an expanded polyglutamine region (82 repeats). Cells in the rodent striatum began to express inclusions of mutant htt protein as early as 1 week after lentiviral transduction. The number and size of the inclusions increased progressively during the 4 weeks after injection. Neuronal degeneration and loss of spiny neurons was observed in the injected striatum ((2002) J Neurosci. 22:3473-3483). This invention uses immune deficient mice and will, for the first time, allow efficacy testing for human stem cell therapies to treat HD.

In one aspect this invention provides an isolated mesenchymal stem cell for delivering a siRNA, miRNA or dsRNA polynucleotide into a target cell comprising, or alternatively consisting essentially of, or yet further consisting of, an exogenous DNA sequence expressing the siRNA, miRNA or dsRNA polynucleotide and which delivers the siRNA, miRNA or dsRNA polynucleotide to the target cell via cellular protrusion or a microvesicle. In a further aspect, the polynucleotide and/or siRNA, miRNA or dsRNA is delivered to the target cell by any method which excludes a gap junction via connexin. In one aspect, the isolated mesenchymal stem cell is placed in communication with the target cell under conditions suitable for transfer of the siRNA, miRNA or dsRNA polynucleotide to the target cell via a cellular protrusion or a microvesicle.

Also provided is a mesenchymal stem cell comprising, or alternatively consisting essentially of, or yet further consisting of, an exogenous siRNA, miRNA or dsRNA sequence or alternatively or in combination with a DNA sequence encoding a siRNA, miRNA or dsRNA sequence. Also provided is a mesenchymal stem cell comprising, or alternatively consisting essentially of, or yet further consisting of, an exogenous DNA sequence encoding a siRNA, miRNA or dsRNA sequence alone or in combination with the siRNA, miRNA or dsRNA sequence. In a further aspect, each of the MSC described above can establish a cellular protrusion with a target cell thereby delivering the polynucleotide and/or the siRNA, miRNA or dsRNA to the target cell. In a further aspect, the MSC can deliver the polynucleotide and/or the siRNA, miRNA or dsRNA or the polynucleotide encoding it via a microvesicle to the target cell. In a further aspect, the polynucleotide and/or siRNA or dsRNA is delivered to the target cell by any method which excludes a gap junction via connexin. In one aspect, the mesenchymal stem cell is an isolated mesenchymal stem cell and in another aspect the cell is present in tissue isolated from a suitable subject, such as lipoaspirate or bone marrow sample.

A MSC of the invention may be identified by cell surface markers including, but not limited to, CD90⁺, CD105⁺, CD44⁺, CD73⁺, CD34⁻, CD45⁻.

In a further aspect, the DNA sequence encoding the siRNA, miRNA or dsRNA is integrated into the genome of the MSC. The DNA is operatively linked and incorporated into an expression and/or delivery vector. In a further aspect, the delivery and/or expression vector containing the DNA sequence comprises a promoter that regulates expression of the DNA. A non-limiting example of a promoter is a polymerase-III promoter, such as the H1-RNA gene promoter.

In another aspect, the siRNA, dsRNA or miRNA is directed at a gene mediating a disease such as for example, a genetic disorder, a viral disease or cancer. Non-limiting examples of diseases include Huntington's disease (HD), Parkinson's disease (PD), Alzheimer's disease (AD), acute myocardial infarction (AMI), cystic fibrosis, amyotrophic lateral sclerosis (ALS), age-related macular degeneration (AMD), acute lung injury (ALI), severe acute respiratory syndrome (SARS), acquired immunodeficiency syndrome (AIDS). In a particular aspect, the disease is Huntington's disease and the gene is directed at the mutant Htt gene. An siRNA directed as this gene is 363125_C-16.

Target cells that are recipients of the siRNA, miRNA or dsRNA include without limitation one or more of a nerve cell, a cardiac cell, a lung cell, a muscle cell, a skin cell or a retinal cell. The cell may be of any origin identified as a subject herein, e.g., simian, bovine, canine equine, murine or human.

The cells of this invention can be combined with a carrier such as a solid support, a carrier or a pharmaceutically acceptable carrier. In a further aspect, the composition further comprises a stem cell derived neuron. In a particular aspect, the neuron which is derived from a stem cell selected from the group of a neuroepithelial stem cell, a MSC, an adipose-derived stem cell or an iPSC.

Populations containing a plurality of the cells as described above are further provided. The populations can be substantially homogeneous for the MSC and/or target cell or heterogeneous. Compositions comprising the populations are further provided wherein the populations are combined with a solid support, a carrier or a pharmaceutically acceptable carrier.

The cells and compositions as described above are useful to deliver one or more of a siRNA, miRNA or dsRNA to a target cell by contacting the target cell with the MSC of this invention. Thus, also provided is a method for delivering a siRNA, miRNA or dsRNA polynucleotide into a target cell comprising or alternatively consisting essentially of, or yet further consisting of, contacting the target cell with a mesenchymal stem cell, which mesenchymal stem cell comprises an exogenous DNA sequence expressing the siRNA, miRNA or dsRNA polynucleotide, thereby delivering the siRNA or dsRNA polynucleotide to the target cell. The MSC can be delivered alone or in combination with a pharmaceutically acceptable carrier. Without being bound by theory, the delivery can independently or in combination occur by or through a cellular protrusion and/or a microvesicle. In a further aspect, the polynucleotide and/or siRNA or dsRNA is delivered to the target cell by any method which excludes a gap junction via connexin. In one aspect, the mesenchymal stem cell is an isolated mesenchymal stem cell and in another aspect the cell is present in tissue isolated from a suitable subject, such as lipoaspirate or bone marrow sample.

Also provided is a method for treating a genetic condition mediated by the presence of a mutated allele in a subject, for example Huntington's disease in a patient by administering to the patient the MSC as described above or a composition comprising, or alternatively consisting essentially of, or yet further consisting of, a mesenchymal stem cell, wherein the polynucleotide and/or the siRNA, miRNA or dsRNA is directed at RNA encoded by a mutant Htt gene, and can deliver the siRNA, miRNA or dsRNA to a target nerve cell in the patient. Without being bound by theory, in one aspect, the MSC of the invention is one in which the polynucleotide and/or siRNA, miRNA or dsRNA is independently or collectively delivered through a cellular protrusion and/or a microvesicle, thereby treating the disease. In one aspect, the mesenchymal stem cell is an isolated mesenchymal stem cell and in another aspect the cell is present in tissue isolated from a suitable subject, such as lipoaspirate or bone marrow sample.

In another aspect, the DNA encodes siRNA directed to a mutant Htt gene, an example of which is 363125_C-16. The target cell can be a neuron or a stem cell derived neuron which can be derived from one or more of a neuroepithelial stem cell, a MSC, an adipose-derived stem cell or an iPSC.

Subjects treated by this method include a simian, a bovine, an equine, a canine, a murine or a human patient.

In some embodiments, provided is a mesenchymal stem cell comprising, or alternatively consisting essentially of, or yet further consisting of an exogenous siRNA, dsRNA, or miRNA sequence alone or in combination with an exogenous DNA sequence encoding a siRNA, dsRNA, or miRNA sequence, wherein the mesenchymal stem cell can deliver the sequence and/or polynucleotide encoding the sequence to a target cell. Without being bound by theory, in one aspect the MSC establishes a cellular protrusion with a target cell thereby delivering the polynucleotide and/or siRNA. miRNA or dsRNA. In other aspect they are delivered by a microvesicle to the to the target cell. In a further aspect, the polynucleotide and/or siRNA, miRNA or dsRNA is delivered to the target cell by any method which excludes a gap junction via connexin.

A MSC of the invention may be identified by cell surface markers including, but not limited to, CD90⁺, CD105⁺, CD44⁺, CD73⁺, CD34⁻, CD45⁻.

In one aspect, the DNA sequence is integrated into the genome of the mesenchymal stem cell. In another aspect, the DNA sequence further comprises an expression or delivery vector. In another aspect, the expression or delivery vector is a lentiviral vector. In yet another aspect, the vector comprises a promoter regulating expression of the dsRNA, miRNA or siRNA. In one aspect, the promoter is a polymerase-III H1-RNA gene promoter. In one aspect, this method provides for the DNA sequence to be integrated into the genome of the mesenchymal stem cell.

To generate the cell, a mesenchymal stem cell is obtained or isolated from a suitable tissue or other source, e.g., created from a differentiated embryonic stem cell or iPSC. The siRNA, dsRNA, or miRNA is prepared using chemical or other methods and can be passively transferred into the stem cell by co-culture with SID-1 DNA or the siRNA, dsRNA, or miRNA can be inserted into a suitable vector such as the lentiviral vector described herein with the appropriate regulation sequences. The cell population, after insertion of the siRNA, dsRNA, or miRNA, can be expanded or differentiated as appropriate.

In some embodiments, the siRNA is directed at a gene mediating a disease. In one aspect, the disease is selected from the group consisting of genetic disorder wherein the diseased is caused by the presence of a mutated allele, viral infection or disease, and cancer or other neoplasm. In another aspect, the disease is selected from the group consisting of Huntington's disease (HD), Parkinson's disease (PD), Alzheimer's disease (AD), acute myocardial infarction (AMI), cystic fibrosis, amyotrophic lateral sclerosis (ALS), age-related macular degeneration (AMD), acute lung injury (ALI), severe acute respiratory syndrome (SARS), acquired immunodeficiency syndrome (AIDS), and others. When the disease is Huntington's disease, the siRNA, dsRNA or miRNA can be directed at single nucleotide polymorphisms adjacent to the CAG repeats mutant Htt gene, or any mutant Htt gene, or a single siRNA. dsRNA or miRNA directed at multiple mutant forms of the Htt gene. In one aspect, the siRNA is 363125_C-16.

In some embodiments, the target cell for the mesenchymal stem cell is selected from the group consisting of a nerve cell, a cardiac cell, a lung cell, a muscle cell, a skin cell, and a retinal cell, among others.

In some embodiments, the mesenchymal stem cell is of mammalian origin. In some embodiments, the mammalian origin is simian, bovine, equine, murine or human. In an alternate embodiment, the mammalian origin is human. Methods to isolate such cells are known in the art and have been published by the Applicants.

In some embodiments, the mesenchymal stem cell is combined with a stem cell derived neuron or other cell, such as for example, a neuroepithelial stem cell, a wild-type mesenchymal stem cell, an adipose-derived stem cell, and an induced pluripotent stem cell for use in the method or compositions.

In one aspect, the mesenchymal stem cell for insertion of the siRNA, dsRNA, or miRNA is an isolated mesenchymal stem cell from all other cellular components or alternatively, only isolated from the host, i.e., still contained within the tissue. In one aspect, this invention provides the MSC of this invention and other cells necessary for clonal propagation or expansion or tissue-specific differentiation. Thus, in another aspect, this invention provides an expanded or differentiated population created by growing or culturing the MSC of this invention under appropriate conditions to obtain the population of cells, each cell having inserted therein the siRNA, dsRNA, or miRNA, as was inserted and present in the MSC from which the population originated.

Also provided is a population of mesenchymal stem cells of this invention that are clonally derived and therefore substantially homogeneous. Methods to clonally expand MSC are known in the art. In another aspect, the invention provides methods to expand nonclonal populations of mesenchymal stem cells of this invention and to differentiate them to the appropriate tissue type, by growing the MSC under suitable conditions that provide for differentiation and expansion. Such general methods are known in the art.

Also provided is an expanded clonal or differentiated population of mesenchymal stem cells of this invention.

Also provided is a composition comprising a mesenchymal stem cell of this invention, a population of mesenchymal stem cells of this invention, or an expanded population of mesenchymal stem cells of this invention, and a carrier. In some embodiments, the carrier is a pharmaceutically acceptable carrier as described above.

Also provided is a method for delivering a siRNA, dsRNA or miRNA polynucleotide into a target cell comprising, or alternatively consisting essentially of, or yet further consisting of contacting the target cell with any one or more of a MSC, a population comprising, or alternatively consisting essentially of, or yet further consisting of, the MSC (clonal or differentiated) mesenchymal stem cell, which mesenchymal stem cell comprises an exogenous DNA sequence expressing the siRNA, dsRNA or miRNA polynucleotide, thereby delivering the siRNA, dsRNA or miRNA polynucleotide to the target cell through a cellular protrusion.

A MSC may deliver the siRNA, dsRNA or miRNA to the target cell through a cellular protrusion. In one aspect, the cellular protrusion is a cytoplasmic extension. In another aspect, the cellular protrusion is a tunneling nanotubule. In yet another aspect, the cellular protrusion is selected from the group consisting of a broad area of cellular contact, a thin, elongated, active filopodia or lamellipodia, a cytonemes, a cytoneme-like protrusion, an apical peripodial extension, a myopodia, a myopdia-like protrusion, a cellular extension, or an apical or lateral cell protrusion.

Also provided is a method for delivering a siRNA, dsRNA or miRNA polynucleotide into a target cell comprising, or alternatively consisting essentially of, or yet further consisting of placing the target cell in communication with any one or more of a MSC, a population comprising, or alternatively consisting essentially of, or yet further consisting of, the MSC (clonal or differentiated) mesenchymal stem cell under conditions suitable for transfer of the siRNA, dsRNA, or miRNA polynucleotide to the target cell via a microvesicle, which mesenchymal stem cell comprises an exogenoDNA sequence expressing the siRNA, dsRNA, or miRNA polynucleotide, thereby delivering the siRNA, dsRNA, or miRNA polynucleotide to the target cell via the microvesicle.

Communication between a MSC and a target cell can be culture medium, biocompatible scaffold for cell growth, or a body such as an animal body or a human body. Accordingly, a MSC can be placed in the culture medium of the target cell so that a microvesicle secreted by the MSC can travel to the target cell and deliver the siRNA, dsRNA, or miRNA to the target. A MSC can also be placed on any platform suitable for cell growth, differentiation or migration on which movement of a microvesicle between a MSC and a target cell is not restricted. In some embodiments, the MSC is placed in a body containing the target cell, where the MSC can migrate to the proximity of the target cell and deliver the polynucleotide to the target cell via a microvesicle.

Conditions suitable for transfer of a siRNA, dsRNA or miRNA polynucleotide from a MSC to a target cell via a microvesicle refers to conditions suitable for cell growth or migration. Examples of suitable conditions for stem cells to deliver a polynucleotide to a target cell include Yuan et al. (2009) PLoS ONE, 4(3):e4722, which is incorporated by reference in its entirety, and those outlined in the foregoing paragraph.

Microvesicles are shed from many cell types under a variety of situations, often due to activation or apoptosis, but also as a normal function of their activities. Embryonic tem cells have been reported to transfer miRNA to neighboring cells by microvesicles (Yuan et al. (2009) PLoS ONE, 4(3):e4722). Applicants have observed that MSCs in normal cultures shed microvesicles containing siRNA.

In another aspect, the DNA sequence further comprises an expression or delivery vector. In another aspect, the expression or delivery vector is a lentiviral vector. In yet another aspect, the vector comprises a promoter regulating expression of siRNA. In one aspect, the promoter is a polymerase-III H1-RNA gene promoter.

In some embodiments, the siRNA is directed at a gene mediating a disease. In one aspect, the disease is selected from the group consisting of genetic disorder, viral disease, and cancer. In another aspect, the disease is selected from the group consisting of Huntington's disease (HD), Parkinson's disease (PD), Alzheimer's disease (AD), acute myocardial infarction (AMI), cystic fibrosis, amyotrophic lateral sclerosis (ALS), age-related macular degeneration (AMD), acute lung injury (ALI), severe acute respiratory syndrome (SARS), acquired immunodeficiency syndrome (AIDS), and others. In one aspect, the disease is Huntington's disease. In one aspect, the siRNA is directed at a SNP adjacent to the CAG repeats in the mutant Htt gene. In a particular aspect, the siRNA is 363125_C-16.

In some embodiments, the target cell is selected from the group consisting of a nerve cell, a cardiac cell, a lung cell, a muscle cell, a skin cell, and a retinol cell.

In some embodiments, the mesenchymal stem cell is of mammalian origin. In some embodiments, the mammalian origin is simian, bovine, murine or human.

In some embodiments, the mesenchymal stem cell is co-administered with a stem cell derived neuron or other stem cell type. In one aspect, the stem cell is selected from the group consisting of a neuroepithelial stem cell, a mesenchymal stem cell, an adipose-derived stem cell, and an induced pluripotent stem cell.

In one aspect, the mesenchymal stem cell is an isolated mesenchymal stem cell.

The method can be practiced in vitro, in vivo, or ex vivo. When practiced in vitro, the MSC or compositions containing the MSC of this invention are contacted with a culture of the target cell under conditions that allow for the transfer of the RNA into the target cell. In vitro practice of the method provides a screen for alternative methods and small molecules. Alternatively, the method is practiced ex vivo by taking a primary cell culture and co-culturing the cells under appropriate conditions. Ex vivo the method is useful to test the therapy prior to administration to a subject such as a human patient. In vivo the method can be practiced to produce an animal model to assay or treat as subject also as provided herein.

When practice in vivo, the method can be used to treat Huntington's disease in a subject such as a human patient by administering to the patient the MSC alone or in combination with other factors. The MSC is administered by direct injection into the tissue to which the RNA is to be transferred. For example the MSC can comprise an exogenous DNA sequence encoding a siRNA, dsRNA, or miRNA sequence directed at a mutant Htt gene, and can deliver the siRNA, dsRNA, or miRNA to a target nerve cell in the subject through a cellular protrusion, thereby treating the disease.

In some embodiments, the method further comprises administering to the patient a stem cell derived neuron. In one aspect, the stem cell derived neuron is administered prior to or after administration of the mesenchymal stem cell. In another aspect, the stem cell derived neuron is administered together with the mesenchymal stem cell.

In some embodiments, the stem cell is selected from the group consisting of a neuroepithelial stem cell, a mesenchymal stem cell, an adipose-derived stem cell, and an induced pluripotent stem cell.

In some embodiments, the administering comprises injecting to the brain or other CNS tissue. In some embodiments, the administering comprises intravenous injection, or direct injecting into the spinal cord, distal or proximal to the side of the target cell.

In a specific embodiment, the subject for the method is an equine, a bovine, a simian, a canine or a human patient. In a more specific embodiment, the subject is a human patient.

Also provided is a method for delivering a siRNA, dsRNA, or miRNA polynucleotide to the brain of a patient across the blood brain barrier, comprising administering a mesenchymal stem cell to the patient, which mesenchymal stem cell comprises an exogenous DNA sequence expressing the siRNA, dsRNA, or miRNA polynucleotide, thereby delivering the siRNA, dsRNA, or miRNA polynucleotide to a target cell in the brain through a cellular protrusion.

In one aspect, the administering comprising intravenous injection, injecting into the brain, or injecting into the spinal cord, distal or proximal to the side of the target cell.

Also provided is a method for determining if expression of a test gene is required for a cellular function comprising contacting a test cell with a mesenchymal stem cell, which mesenchymal stem cell comprises an exogenous DNA sequence encoding a siRNA, dsRNA, or miRNA sequence directed at the test gene, thereby delivering the siRNA, dsRNA, or miRNA polynucleotide to the test cell through a cellular protrusion, wherein disruption of the cellular function indicates that expression of the test gene is required for the cellular function.

Also provided is a kit for delivering a siRNA, dsRNA, or miRNA polynucleotide into a target cell, comprising a mesenchymal stem cell comprising an exogenous DNA sequence expressing the siRNA, dsRNA, or miRNA polynucleotide wherein the mesenchymal stem cell can establish a cellular protrusion and/or microvesicle with the target cell thereby delivering the siRNA, dsRNA, or miRNA to the target cell, and instructions for use in delivering the siRNA, dsRNA, or miRNA. Target cells are as described above. The kit may further comprise a gene delivery vector as described herein and/or instructions for use.

### EXPERIMENTAL EXAMPLES

### Example 1

### MSC Infuses siRNA to Target Cells

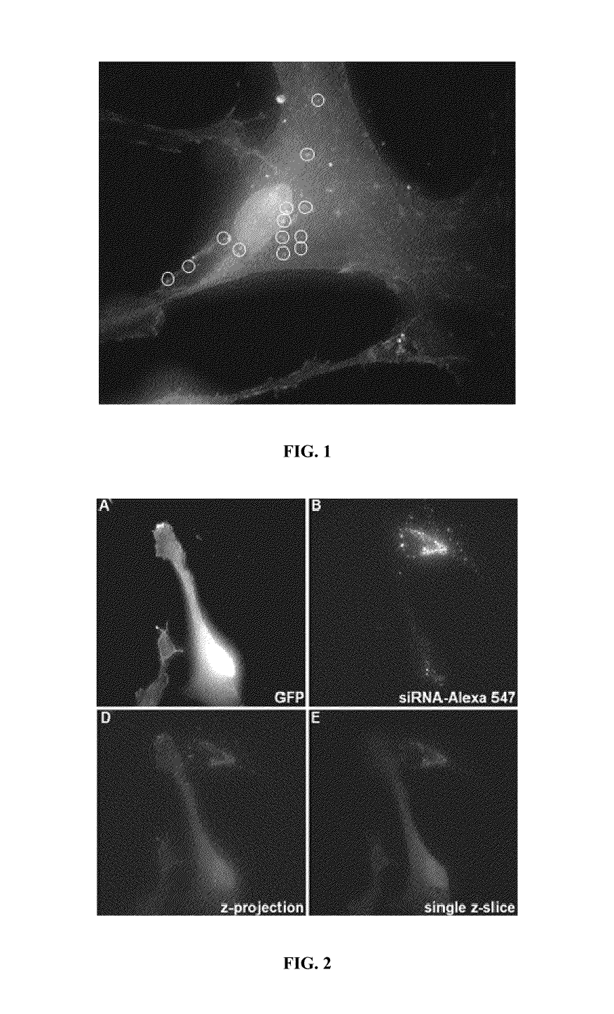

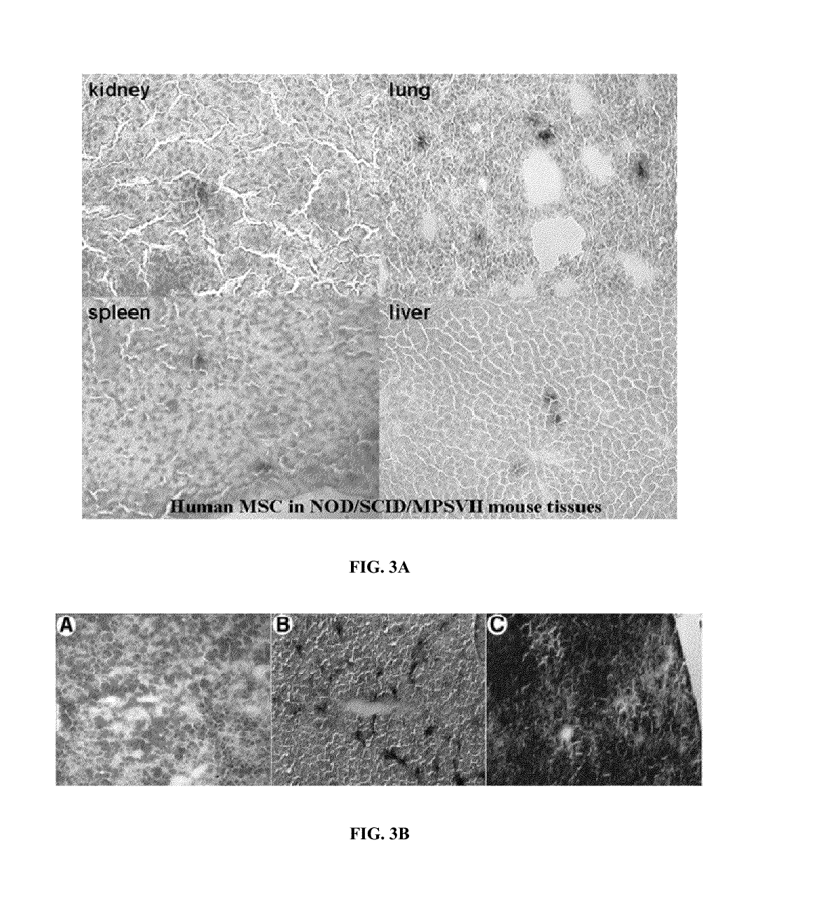

It is discovered that MSC can be used deliver siRNA robustly into damaged cells in vivo. FIG. 1 shows an eGFP-labeled MSC that has had alexa-fluor-labeled anti mutant htt siRNA (red) transferred into it from an adjacent, non-GFP MSC (see also FIGS. 3A-3B). Brighter spots have coalesced into lysosomes after transfer, but smaller siRNA amounts are scattered throughout the cytoplasm and nucleus. Human mesenchymal stem cells (MSC) can be transduced to produce siRNA and other RNA-modifying moieties (siRNA/ miRNA hybrids and others), to reduce levels of mutant htt RNA and protein levels in neurons.

It is discovered that MSC will readily transfer the small RNA molecules directly through cell-to-cell contact. The cell-to-cell contact may include cellular protrution, cytoplasmic extension, or tunneling nanotubes. It has been demonstrated that MSC's rapidly home to the site of injury or distress in the body. MSC's survive integrated into the tissues of immune deficient mice for up to 18 months, and produce the products of introduced transgenes for this duration. See, e.g., Dao et al. (1997) Stem Cells. 15:443-454; Meyerrose et al. (2007) Stem Cells. 25:220-227; Meyerrose et al. (2008) Stem Cells. 26:1713-1722; Nolta et al. (1994) Blood. 83:3041-3051; Tsark et al. (2001) J Immunol. 166:170-181; Wang et al. (2003) Blood. 101 (10) 4201-4208; Bauer et al. (2008) Mol Ther. 16:1308-1315; Rosova et al. (2008) Stem Cells 26:2173-2182; and Wu et al. (2003) Transplantation. 75:679-685. In a decade-long study that, after genetic modification and transplantation, MSC's have been shown to be safe and do not cause adverse events or tumors (Bauer et al. (2008) Mol Ther. 16:1308-1315). The current delivery strategy shows that, in addition to secretion of protein products, small interfering RNA can be directly secreted from MSC into target cells through cell-to-cell contact (FIG. 1, FIG. 2). In addition to the trophic effects of MSC on repairing damaged neurons, could have a significant impact on the severity of HD progression.

It is also discovered that MSC can transfer small RNA moleculars through microvesicles secreted by the MSCs. It is shown in FIG. 1 that siRNA appeared in microvesicles outside the cells, as indicated by the white circle outside the cells. Therefore, MSCs may deliver siRNA to target cells either by a direct cell-to-cell contact such as cellular protrusion, or by indirect transfer through microvesicles secreted by the MSCs.

### Example 2

### MSC Isolation and Transduction

Human MSC can be collected from normal donors and expanded under clinically relevant conditions. Applicants have previously demonstrated that human MSC readily uptake viral vectors (see, e.g., Dao et al. (1997) Stem Cells. 15:443-454; Meyerrose et al. (2007) Stem Cells. 25:220-227; Meyerrose (2008) Stem Cells. 26:1713-1722; and Nolta (1994) Blood. 83:3041-3051). Lentiviral vectors have been developed to express several different forms of the mutant htt protein for direct injection into the left and right striata, for development of an HD mouse on the permissive xenograft background. Coding sequences in these vectors included the Htt cDNA coding for amino acids 1-400 with CAG repeat lengths of 18 (wild-type, normal gene), 44, and 82. Introduction of the gene with 82 repeats caused rapid onset of inclusion formation and behavioral deficit when introduced in rodents using the viral vector strategy as described, with a 1-3 week delay caused by the gene with 44 repeats (DiFiglia et al. (2007) Proc Natl Acad Sci USA. 104:17204-17209).

### Example 3

### Allele-Specific siRNA

The goal of siRNA knockdown in HD is to suppress the mutant protein while sparing mRNA transcribed from the normal allele. Schwarz et al., in 2006, first demonstrated that allele-specific suppression of huntingtin mRNA expression was possible (Schwarz et al. (2006) PLoS Genet. 2:e140). van Bilsen et al. demonstrated allele-specific suppression of endogenous huntingtin gene expression in cells isolated directly from Huntington's disease patients (van Bilsen et al. (2008) Hum Gene Ther. 19:710-719). van Bilsen et al. have determined SNP sites to target that are located remotely from the CAG repeat region. The siRNA known to reduce the mutant gene can be introduced into the mice, directed to SNP rs363125, with 44 CAG codons versus 19 CAG codons on the wild-type allele. A vector to express the sequence identical to siRNA 363125_C-16 as tested in van Bilsen's study has been created and a specific siRNA vector for the 82 repeat Htt allele can be utilized. It is also contemplated that a siRNA vector directed to various mutant forms of the Htt gene can be used which can be used for most patients.

### Example 4

### Assessment of siRNA Transfer

Transfer of the siRNA has been done in vitro, using fluorescence-tagged synthetic siRNA. These initial studies used the anti-htt 150 sequence. An siRNA vector can also be prepared as follows: the backbone for the Htt SiRNA is pCCLc-X, with the H1 promoter (from the pSuper vector from Oligoengine) cloned in the “X” position, driving the siRNA (ex pCCLc-Hlp-Htt150 siRNA). U87 cells, dermal fibroblasts, neural stem cells, a rapidly growing tumor line isolated from NOD/SCID/MPSVII mice, and HD patient iPS cells can be used to develop HD neural stem cells. Normal cells can have the mutant human htt allele transferred into them and can act as recipient cells to test the efficiency of MSC-mediated siRNA transfer and protein knockdown in vitro. Donor and target cells can be separated cleanly by FACS based on cell surface markers that differ between MSC and neural cells, or by GUSB expression, and can then be tested by FACS (for fluorescent siRNA transfer) and by quantitative RT PCR, western blot, and microassay for protein levels. In all studies, knockdown of the eGFP protein can be done by MSC-mediated transfer of the anti-eGFP siRNA, as a positive control easily monitored by FACS.

### Example 5

### siRNA Transfer From MSC into Target Cells

Transfer of the alexa-fluor labeled anti-mutant htt siRNA from MSC into target cells has been examined. The htt siRNA used was siRNA Htt150, originally described in DiFiglia et al. (2007) Proc Natl Acad Sci USA. 104:17204-17209. The rate of transfer was directly visualized (FIG. 2). Equipment used was the Deltavision deconvolution microscope, using a 60× objective and taking 60 planes at 0.2 micron-steps. These images can also be rotated to ensure that siRNA have been transferred into the cell, and are not at the surface. Rate of the direct cell-to cell transfer of labeled siRNA by MSC has been examined. The methods used to generate the data in FIG. 2 can be used to test in vitro transfer efficacy of each new siRNA and siRNA/miRNA hybrid construct. FACS analysis can determine the degree of transfer from donor to target cells, and the percentage knockdown of the eGFP protein by MSC-delivered anti-eGFP siRNA continually produced by lentiviral transduction can be assessed. Reduction of eGFP levels can be assessed using FACS of target neural cells.

### Example 6

### HD Model to Test Human Stem Cell Therapies in Vivo

To generate the mouse model, NOD/SCID/MPSVII and NOG immune deficient mice can be injected with lentiviral vectors coding for either the mutant or wild type htt protein, into the right and left striata. The mice will be anesthetized and then a small incision will be made in the scalp, providing room to drill a 1 mm burr hole in the animal's skull. The mutant or wild-type lentivirus will be injected into the striatum at a controlled rate as described by de Almeida, et al. (de Almeida et al. (2002) J Neurosci. 22:3473-3483). Sets of 12 mice will be done in each experiment, 4 per arm, and repeated eight times with MSC from different donors. Following the injection, bone wax will be placed over the burr hole to control bleeding, and the scalp over the hole will be closed with small sutures. Starting at one week after the injection of the virus, behavioral effects will be assessed. Prior to surgery, the mice will be trained to walk across a beam to a box. The beam will be lined with paper so that the feet of the mice can be stained with ink, enabling assessment of behavioral defects demonstrated in their footfall patterns. Four weeks after the initial injections the animals will be transplanted with siRNA-producing MSC vs. scrambled siRNA-producing MSC using the same intra-striatal injection technique. Again, the beam test will begin one week post-op, to look for changes to the mice's gait. Proper sham controls and vectors expressing scrambled siRNA will be used to ensure that any changes noted are due to treatment and not effects of the surgeries themselves. The mice will be sacrificed at various time points and their brains harvested for assessments, as described further below.

### Example 7

### Human MSC Re-Capturing From Mouse Brains

Human MSC can be re-captured from the brain tissue after specified timepoints using GUSB FACS sorting. This sorting strategy allows to separate the living cells recovered from the brain into GUSB positive (human) and negative (murine) cells, to assess levels of siRNA in each. Human donor GUSB+ cells will be viably isolated from the mouse brain by FACS, using the diffusible substrate. The number and percentage of cells migrating into the injured area of tissue in each assay can be rapidly quantitated using the NOD/SCID/MPSVII mice. The use of the GUSB- based flow assay coupled with cell surface analysis for murine MHC will confirm that enzyme has not been taken up the bystander effect or by host macrophages engulfing dying cells. The enzymatic labeling is quite specific, and although the released enzyme can be taken up by neighboring cells, it is in a processed form no longer detectable by the histochemical or FACS-based analyses (Sands et al. (1997) Neuromuscul Disord. 7:352-360; Wolfe et al. (1992) Nature. 360:749-753). This will be verified for each cell population to be tested. Cells recovered from the brain will be assessed for alterations in htt proteins and mRNA levels, using quantitative real time PCR and protein analyses. Using the NOD/SCID/MPSVII model, human cells from the mouse tissues can be viably sorted, based on the lipophilic substrate for the GUSB enzyme. They can also be sorted using CD105 on human MSC.

The captured MSC can then be cultured in single colony assay, to ensure intact genetic content, or taken immediately for chromosome spreads and FISH (Wang et al. (2003) Blood. 101 (10) 4201-4208). The GUSB+ cells will be isolated, using Influx cytometer, from single cell suspensions from the brain. Applicants have been able to recover up to 20% GUSB+ human cells from the liver after injury, and 5% from the muscle in hindlimb ischemia. Adequate levels has also been recovered from the brain after transplantation. The isolated numbers are adequate for all assays. Cells that had delivered siRNA into the brain will be recovered and will be assessed for changes in htt protein levels. Approximately 10,000 cells per assay are required for the best analyses, and fewer can be used.

Any adverse events will be closely examined, such as ectopic aberrant tissue differentiation or tumor formation occurring in the brains of the mice from human MSC in vivo, in the proposed studies, as has been reported (Bauer et al. (2008) Mol Ther. 16:1308-1315). The immune deficient mouse studies with human marrow and adipose derived MSC will be conducted under GLP (Good Laboratory Practice) conditions as mandated by the FDA, so that they can be directly translational for MSC-based tissue repair therapies.

### Example 8

### Mesenchymal Stem Cell Engineering and Transplantation

It has been demonstrated in Applicants' earlier studies that MSC's represent a population of stem cells that are easily obtained and very amenable to either lentiviral or retroviral transduction, making them an excellent avenue for cell-based therapies involving a wide range of end tissue targets. Evidence of vector silencing has not been observed, and sustained and safe in vivo expression of transgene products for up to 18 months (duration of the experiment) have been reported (Dao et al. (1997) Stem Cells. 15:443-454; Meyerrose et al. (2007) Stem Cells. 25:220-227; Meyerrose et al. (2008) Stem Cells. 26:1713-1722; Nolta et al. (1994) Blood. 83:3041-3051).

### Example 9

### Quantitation of Human MSC in Vivo to Demonstrate Feasibility of Adequate Cell Recovery to Determine siRNA Effectiveness

A duplex qPCR system was used to enumerate the contribution of human MSC per organ through simultaneous detection of the murine rapsyn and human B-globin genes (Meyerrose et al. (2007) Stem Cells. 25:220-227; Meyerrose et al. (2008) Stem Cells. 26:1713-1722). As little as 0.005 ng of either species' DNA was detected within 100 ng of total DNA from the alternate species. MSC migrated into the brain after intravenous injection, and were still present six months later. Absolute human donor cell contribution per organ was calculated as described to estimate total persisting MSC (Meyerrose et al. (2007) Stem Cells. 25:220-227; Meyerrose et al. (2008) Stem Cells. 26:1713-1722). With direct injection into the brain the cells are expected to be present in more robust numbers and to migrate readily throughout the tissue. It is also contemplated that MSC can be injected into the spinal cord. Following injection into the spinal cord, distal or proximal to the target site in the brain, MSC can migrate to the target site and deliver siRNA to the target site.

### Example 10

### Improved Immune Deficient Mouse Model For Enhanced Detection of Human Cells

Mucopolysaccharidosis Type VII (MPSVII) is caused by a deficiency in B-glucuronidase (GUSB) activity. The NOD/SCID/MPSVII strain allows rapid visualization of human cells which carry normal levels of the enzyme beta-glucuronidase, against the background mouse tissues which are null for the enzyme. An example of the ease and specificity of locating transplanted human stem cells in murine tissue sections is shown in FIG. 3. This strain has been used to pinpoint the areas of human stem cell-mediated tissue repair in damaged organs (Meyerrose et al. (2007) Stem Cells. 25:220-227; Meyerrose et al. (2008) Stem Cells. 26:1713-1722; Hess et al. (2008) Stem Cells 26:611-620). Following the enzymatic reaction, slides can be counterstained with antibodies to a tissue-specific protein marker (Hess et al. (2008) Stem Cells 26:611-620; Hofling et al. (2003) Blood 101:2054-2063). The enzymatic stain is quite specific, and although the released enzyme can be taken up by neighboring cells, it is in a processed form no longer detectable by the histochemical analysis (Sands et al. (1997) Neuromuscul Disord. 7:352-360; Wolfe et al. (1992) Nature. 360:749-753). Thus, the individual transplanted human cells stand out vividly against the background, GUSB null murine tissues. Human cells can thus be detected without reliance on expression of cell surface markers or introduced marker genes. A flow cytometric assay also exists to re-isolate the human cells, based only upon GUSB enzyme activity and not cell surface phenotype or other attributes. The novel model of the NOD/SCID MPSVII mouse provides unique opportunities to visualize, track, and recover human cells after transplantation without reliance upon expression of surface proteins or prospective labeling. This system is very useful for recovering MSC from the brains of the mice, for assessment of continued siRNA production over a timecourse, for analysis of genetic integrity for safety studies, and to separate them cleanly from the murine cells to allow a direct measurement of the amounts of mutant vs. normal htt protein in the murine neurons.

### Example 11

### Two-Pronged Cellular Therapy

A two-pronged cellular therapy approach for HD is contemplated. Two cell types can be co-delivered into the neostriatum: spiny neurons generated using hESC technologies, coupled with the MSC therapy to reduce endogenous htt levels. The two-pronged approach can provide a therapy for patients in more advanced stages of the disease, who have lost significant amounts of neural tissue. The MSC will also shelter the transplanted neurons from rejection by the immune system. The two cell types can be co-administered, or one is administered prior to the other.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1-27. (canceled)

28. A method for delivering a siRNA, miRNA or dsRNA polynucleotide into a target cell comprising placing the target cell in communication with a mesenchymal stem cell, which mesenchymal stem cell comprises an exogenous polynucleotide sequence encoding a siRNA, miRNA or dsRNA directed at mediating Huntington's disease, thereby delivering the siRNA, miRNA or dsRNA polynucleotide to the target cell.

29. The method of claim 28, wherein the sequence is delivered through a cellular protrusion and/or via a microvesicle.

30-37. (canceled)

38. The method of claim 28, wherein the siRNA, miRNA or dsRNA is directed at a mutant Htt gene.

39. The method of claim 38, wherein siRNA is 363125_C-16.

40. The method of claim 28, wherein the target cell is a nerve cell.

41. The method of claim 28, wherein the mesenchymal stem cell is of mammalian origin.

42. The method of claim 41, wherein the mammalian origin is simian, bovine, equine, canine, murine or human.

43. The method of claim 41, wherein the mammalian origin is human.

44. The method of claim 28, further comprising administration of a stem cell derived neuron.

45. The method of claim 44, wherein the neuron is derived from a stem cell selected from the group consisting of a neuroepithelial stem cell, a mesenchymal stem cell, an adipose-derived stem cell, and an induced pluripotent stem cell.

46. The method of claim 28, wherein the mesenchymal stem cell is an isolated mesenchymal stem cell.

47. The method of claim 28, wherein the contacting is in vitro, in vivo, or ex vivo.

48. A method for delivering a siRNA, miRNA or dsRNA polynucleotide into a target cell comprising placing the target cell in communication with a mesenchymal stem cell under conditions suitable for transfer the siRNA, miRNA or dsRNA polynucleotide to the target cell via a microvesicle, which mesenchymal stem cell comprises an exogenous polynucleotide sequence encoding the siRNA, miRNA or dsRNA directed at mediating Huntington's disease (HD), thereby delivering the siRNA, miRNA or dsRNA polynucleotide to the target cell via the microvesicle.

49-52. (canceled)

53. The method of claim 48, wherein the target cell is a nerve cell.

54. The method of claim 48, wherein the contacting is in vitro, in vivo, or ex vivo.

55. A method for treating Huntington's disease in a patient comprising administering to the patient a mesenchymal stem cell, which mesenchymal stem cell comprises an exogenous polynucleotide sequence encoding a siRNA, miRNA or dsRNA directed at a mutant Htt gene, and can deliver the siRNA, miRNA or dsRNA to a target nerve cell in the patient through a cellular protrusion and/or via a microvesicle, thereby treating the disease.

56. The method of claim 55, further comprising administering to the patient a stem cell derived neuron.

57. The method of claim 56, wherein the stem cell derived neuron is administered prior to or after administration of the mesenchymal stem cell.

58. The method of claim 56, wherein the stem cell derived neuron is administered together with the mesenchym stem cell.

59. The method of claim 56, wherein the stem cell is selected from the group consisting of a neuroepithelial stem cell, a mesenchymal stem cell, an adipose-derived stem cell, and an induced pluripotent stem cell.

60. The method of claim 55 or 56, wherein the administering comprises injecting to the brain.

61. The method of claim 55 or 56, wherein the administering comprises intravenous injection, or injecting into the spinal cord, distal or proximal to the side of the target cell.

62. The method of claim 55, wherein the patient is a human patient.

63. (canceled)

64. A method for delivering a siRNA, miRNA or dsRNA polynucleotide to the brain of a patient across the blood brain barrier, comprising administering a mesenchymal stem cell to the patient, which mesenchymal stem cell comprises an exogenous polynucleotide sequence encoding the siRNA, miRNA or dsRNA polynucleotide directed at mediating Huntington's disease (HD), thereby delivering the siRNA, miRNA or dsRNA polynucleotide to target cell in the brain through a cellular protrusion and/or via a microvesicle.

65. The method of claim 64, wherein the administering comprising intravenous injection, injecting into the brain, or injecting into the spinal cord, distal or proximal to the side of the target cell.

66-67. (canceled)
